(19) United States
(12) Patent Application Publication
(10) Pub. No.: US 2026/0298806 A1
(43) Pub. Date: Oct. 1, 2026

(54) NATURE-DERIVED AND NATURE-INSPIRED STRUCTURAL COLOR FOR QUANTITATIVE ASSESSMENT OF MATERIAL MICROSTRUCTURE

(71) Applicant: THE REGENTS OF THE UNIVERSITY OF CALIFORNIA, Oakland, CA (US)

(72) Inventors: Lisa Poulikakos, La Jolla, CA (US); Paula Kirya, La Jolla, CA (US)

(21) Appl. No.: 19/480,751

(22) PCT Filed: May 1, 2024
(86) PCT No.: PCT/US2024/027234

§ 371 Date: Oct. 31, 2025

Related U.S. Application Data
(60) Provisional application No. 63/463,518, filed on May 2, 2023.

(51) Int. Cl. G01N 21/21; G01N 33/483; G02B 21/06

(52) U.S. Cl. CPC G01N 21/21; G01N 33/4833; G02B 21/06; G01N 2021/216; G01N 2201/06; G01N 2201/12

(57) ABSTRACT

A method and platform for imaging and analyzing the microstructure of fibrous materials such as biological tissues employ a nature-derived or nature-inspired colorimetric photonic crystal to enhance a polarized light microscopy imaging system. Exemplary colorimetric photonic crystals can be derived from the structural color of an organism, such as a butterfly wing.

### RELATED APPLICATIONS

This application claims the benefit of the priority of U.S. Provisional Application No. 63/463,518, filed May 2, 2023, which is incorporated herein by reference in its entirety.

### FIELD OF THE INVENTION

The present invention relates to a platform and method for visualizing and analyzing microstructure and properties of fibrous materials such as biological tissues. The platform and method employ nature-derived or nature-inspired photonic crystals for material imaging.

### BACKGROUND

Histological imaging techniques are essential for examining tissue structure and identifying pathological conditions. Two common methods are brightfield microscopy and fluorescence microscopy, each with tradeoffs in cost, precision, and invasiveness due to staining processes. Brightfield microscopy is the primary method, involving staining tissue sections with dyes like Hematoxylin and Eosin (H&E) to highlight cellular structures. While it provides qualitative information, it requires extensive preparation time and may lack precision. Fluorescence microscopy, particularly immunohistochemistry (IHC), offers precise identification of specific biological structures but is costly and prone to photobleaching. Stain-free methods like polarimetric imaging, optical coherence tomography, and second harmonic generation microscopy offer alternatives. These methods exploit optical interferometry or the tissues optical anisotropy for characterization without staining, providing high-resolution imaging. However, they require expensive equipment and specialized expertise.

Recent advancements include metasurface-based techniques, offering rapid and noninvasive imaging. Examples include guided-mode resonant dielectric metasurfaces and plasmonically active microscope slides. While promising, these approaches have limitations such as fabrication challenges and restricted tissue depth penetration. Histological imaging techniques vary in their benefits and limitations. Therefore, there is a need for ongoing efforts to improve accuracy, speed, and accessibility in the analysis of microstructure of fibrous materials.

### BRIEF SUMMARY

The disclosure provides a method and platform for imaging and analyzing the microstructure of fibrous materials.

The method provides quantitative assessment of the microstructure of fibrous material. The method includes the steps of mounting the fibrous material on a slide; disposing the slide on top of another slide containing a colorimetric photonic crystal mounted on a microscope stage; imaging the fibrous material using polarized light projected through the slides and an output polarizer; and assessing the intensity profile of the image by a mathematical model.

In one aspect, the colorimetric photonic crystal is derived from the structural color of an organism, such as Morpho butterfly wing.

In one aspect, the fibrous material can be natural fibrous material or synthetic fibrous material. In specific, the fibrous material is a biological material that contains fibrous tissue.

In another aspect, the mathematical model is based on Jones calculus.

The platform employs a polarized light microscopy imaging system enhanced by a natural colorimetric photonic crystal.

In one aspect, the platform provides an enhanced imaging system for the fibrous material. The platform contains a light microscope having a microscope stage and a light source configured to project polarized light through one or more slides disposed on the stage and an analyzing polarizer at the output. The first slide contains a colorimetric photonic crystal; and a second slide retains a sample of the fibrous material. The second slide is disposed on top of the first slide so that when polarized light is projected through the slides, the first slide operates as a birefringent material which alters the incident polarization state of light whereby fiber alignment within the fibrous material is imaged and differentially illuminated.

In one aspect, the fibrous material and photonic crystal are imaged in reflection mode (episcopic); in another aspect the fibrous material and photonic crystal are imaged in transmission mode (diascopic) of the polarized light microscope.

In another aspect, the colorimetric photonic crystal is derived from the structural color of an organism, such as a Morpho butterfly wing.

### BRIEF DESCRIPTION OF THE DRAWINGS

FIGS. 1A-1C are diagrams of an exemplary experimental design according to an embodiment of the inventive approach. FIG. 1A shows a Patient Derived Xenograft model (PDX). Patient derived xenograft (PDX) models of a range of treatment-naïve triple-negative breast cancer (TNBC) subtypes. A group of PDX models were selected based on their stiffness (dense vs. sparse collagen content determined by SHG microscopy and expression of the Epithelial-mesenchymal transition (EMT) transcription factor Twist1). 500,000 PDX cells were implanted into the fourth mammary fat pads of immunodeficient mice, corresponding to Institutional Animal Care and Use Committee (IACUC) guidelines for animal care. When tumors reached 1000 mm³, tumor tissues were collected, fixed with 4% Paraformaldehyde at 4° C. for 48 h and embedded in paraffin. These fixed paraffin embedded tissues were sectioned at 3 μm thickness. FIG. 1B shows the preparation of the butterfly wing section. A ~1″×1″ section a Morpho menelaus butterfly wing was cut out and adhered to a glass slide with clear nail polish. A cover slip was adhered on top to prevent scales from escaping the wing surface. The slide containing the Morpho wing section was placed underneath the slide with the histological tumor section and placed on the rotating stage of the polarized light microscope shown in FIG. 1C. This arrangement was imaged between crossed linear polarizers and the stage was rotated counterclockwise from 0°-180° in 15° increments. Image of Morpho butterfly was taken with an APPLE® iPhone 8 camera. Scanning electron micrographs (SEMs) of the Morpho wing scales were acquired with an FEI Apreo LoVac SEM. Schematic was generated in Biorender. FIG. 1C illustrates a schematic of the Morpho-enhanced polarized light microscopy imaging platform.

FIGS. 2A-2H illustrate correlation of collagen arrangements and intensity profiles. FIGS. 2A-2D correspond to distinct intensity profiles fit to the Jones calculus model (Equation 4) in FIGS. 2E-2H. FIGS. 2A and 2E are for Case 1: ordered and dense collagen arrangements, ↑ R², ↑ δ; FIGS. 2B and 2F are for Case 2: disordered and dense collagen arrangements, ↓ R², ↑ δ. FIGS. 2C and 2G are for Case 3: ordered and sparse collagen arrangements, ↑ R², ↓ δ. FIGS. 2D and 2H are for Case 4: disordered and sparse collagen arrangements, ↓ R², ↓ δ. Jones calculus fit data for each case was simulated in MATLAB®. δ=90° and δ=45° was inserted into the Jones calculus intensity profile for ↑ δ and ↓ δ cases respectively. For 1 R² φ=0 was applied to the intensity profile. To generate a profile with ↓ R², an array of 13 random φ values for φ∈[0°, 45°] was generated with the MATLAB® randi function. This array was then inserted into the Jones fit equation to produce intensities for each 15° of rotation. Following the procedure with experimental data, the MATLAB® Curve Fitter tool was implemented to estimate φ and δ values that produce the best fit of the raw data. The Non-linear least squares method and trust-region algorithm were selected, as well as the constraints φ∈[0°, 180°] and δ∈[0°, 180°]. The R² value of the fit is generated by the curve-fitting tool. The tool arrived at the following values, (e) Case 1: R²=1.0, δ=90.00°±0.063°, (f) Case 2: R²=0.1818, δ=48.048°±76.524°, (g) Case 3: R²=1.0, δ=45.00°±0.000°, (h) Case 4: R²=0.1818, δ=25.46°±38.778°. Collagen Arrangement illustrations were generated in Biorender.

FIGS. 3A-3H illustrate the experimental characterization of the Morpho butterfly wing. FIGS. 3A-3D show polarized light microscope images of the Morpho butterfly wing at varying optical axis orientations between crossed linear polarizers (FIGS. 3A,3B) and for circularly polarized light excitation with a linear analyzer (FIGS. 3C, 3D). FIG. 3E shows the reflectance spectra of the Morpho butterfly wing between crossed linear polarizers at 90° and 150° stage orientations. FIG. 3F illustrates Jones calculus fit (Equation 4) of the Morpho butterfly wing between crossed linear polarizers at varying microscope stage angle orientations. FIG. 3G shows the reflectance spectra of the Morpho butterfly wing for circularly polarized light excitation at 45° and 135° stage orientations. FIG. 3H is a histogram of the color discrimination factor ΔE_{ab} for varying reference stage angles θ in comparison to the orthogonal stage angle orientation at θ+90°.

FIGS. 4A-4G show collagen dense tumor section fiber arrangement Case 1 and Case 2 classification. FIG. 4A shows histological tumor section interfaced with the Morpho butterfly wing section imaged between crossed polarizers in reflectance at varying stage orientation angles 45°, 60°, 75°, 90°. FIG. 4B shows histological tumor section imaged between crossed polarizers in reflectance. FIG. 4C shows the histological tumor section interfaced with the Morpho butterfly wing section imaged between crossed polarizers in reflectance, ROIs representing Case 1 and Case 2 are encircled. FIG. 4D shows the deparaffinized histological tumor section stained with H&E imaged in transmission. FIG. 4E shows SHG micrograph of deparaffinized H&E-stained section taken with the LEICA® SP8 Multiphoton/Confocal System. Images in FIGS. 4B-4D were acquired with a NIKON® Eclipse LV100ND Polarized Light Microscope at 20× magnification at microscope stage rotation of 45°. Scale bars for FIGS. 4A-4E are 50 μm. FIGS. 4F-4G demonstrate Jones calculus fit of average grayscale intensity within encircled ROI at 15° increments of microscope stage rotation from 0°-180° for Case 1 (FIG. 4F) and Case 2 (FIG. 4G). The MATLAB® Curve Fitter tool was implemented to estimate φ and δ values that produce the best fit of the raw data. The non-linear least squares method and trust-region algorithm were selected, as well as the constraints φ∈[−180°, 180°] and δ∈[0°, 180°]. The R² value of the fit was generated by the curve-fitting tool. The δ and R² values for Case 1 and Case 2 were extracted from the MATLAB® Curve Fitter tool.

FIGS. 5A-5G show collagen sparse tumor section fiber arrangement characterization Case 3 and Case 4 classification. FIG. 5A shows the histological tumor section interfaced with the Morpho butterfly wing section imaged between crossed polarizers in reflectance at varying stage orientation angles 45°, 60°, 75°, 90°. FIG. 5B shows the histological tumor section imaged between crossed polarizers in reflectance. FIG. 5C shows the histological tumor section interfaced with the Morpho butterfly wing section imaged between crossed polarizers in reflectance, ROIs representing Case 3 and Case 4 are encircled. FIG. 5D shows the deparaffinized histological tumor section stained with H&E imaged in transmission. FIG. 5E shows SHG micrograph of deparaffinized H&E-stained section taken with the Lecia SP8 Multiphoton/Confocal System. Images in FIGS. 5A-5C were acquired with a NIKON® Eclipse LV100ND Polarized Light Microscope at 20× magnification and were taken at microscope stage rotation of 45°. Scale bars for a-e are 50 μm. FIGS. 5F-5G demonstrate Jones fit of average grayscale intensity within encircled ROI at 15° increments of microscope stage rotation from 0°-180° for Case 3 (FIG. 5F) and Case 4 (FIG. 5G). The MATLAB® Curve Fitter tool was implemented to estimate φ and δ values that produce the best fit of the raw data. The non-linear least squares method and trust-region algorithm were selected, as well as the constraints φ∈[−180°,180°] and δ∈[0°,180°]. The R² value of the fit was generated by the curve-fitting tool. The δ and R² values for Case 3 and Case 4 extracted from the MATLAB® Curve Fitter tool.

### DETAILED DESCRIPTION OF EMBODIMENTS

Fibrous materials play a vital role in various fields, including textiles, construction, biomedicine, and aerospace, owing to their diverse properties and applications. Fibrous materials are substances composed of long, thread-like structures, often characterized by their high tensile strength and flexibility. These materials are prevalent in both natural and synthetic forms, serving various functions across industries and biological systems.

### Natural Fibrous Materials

Collagen is found abundantly in connective tissues like tendons, ligaments, and skin, collagen provides structural support and elasticity to the body. It forms the basis of many biomedical applications, including wound dressings and tissue engineering scaffolds.
Cellulose has the primary component of plant cell walls, cellulose fibers are widely used in textiles, papermaking, and biodegradable plastics. They offer excellent strength and durability.
Silk is produced by silkworms and spiders, silk fibers are known for their exceptional tensile strength and luxurious texture. They are used in textiles, medical sutures, and even electronics due to their biocompatibility.
Wool is derived from sheep, wool fibers possess unique properties such as insulation, moisture-wicking, and flame resistance. They are commonly used in clothing, carpets, and upholstery.

### Synthetic Fibrous Materials

Nylon: A synthetic polymer known for its strength and elasticity, nylon fibers are used in a wide range of applications, including textiles, ropes, and engineering components.
Polyester: Versatile and durable, polyester fibers are extensively used in clothing, home furnishings, and industrial applications due to their resistance to wrinkles, shrinking, and abrasion.
Carbon Fiber: Made from carbon atoms aligned in a crystal structure, carbon fibers offer exceptional strength-to-weight ratio and stiffness. They are used in aerospace, automotive, and sporting goods industries.
Fiberglass: Composed of glass fibers embedded in a resin matrix, fiberglass is lightweight, corrosion-resistant, and electrically insulating. It finds applications in construction, marine, and automotive industries.

### Biological Fibrous Materials

Muscle Fibers: Comprising proteins like actin and myosin, muscle fibers enable contraction and movement in animals. They are crucial components of muscle tissues.
Neural Fibers: Nerve cells or neurons transmit electrical impulses through long, slender projections called axons. These fibers facilitate communication within the nervous system.
Extracellular Matrix (ECM): In biological tissues, the ECM consists of fibrous proteins like collagen, elastin, and fibronectin. It provides structural support, regulates cell behavior, and facilitates tissue regeneration.

Analyzing biological fibrous materials involves examining various aspects to understand their structure, properties, and functions. For example, structure and morphology study the organization, arrangement, and morphology of the fibers at different length scales, from the molecular level to the macroscopic level. Methods such as microscopy can provide insights into fiber structure. Biocompatibility is one aspect to assess the compatibility of the fibers with biological systems, including cell adhesion, proliferation, and tissue response. In vitro cell culture studies and in vivo animal models can be employed to evaluate biocompatibility. Biological functionality aims to investigate the functional role of the fibers in biological processes such as tissue development, wound healing, and cell signaling. This may involve studying how fibers interact with cells, extracellular matrix components, and other biomolecules. Study of degradation and biodegradability is to understand the degradation kinetics and biodegradability of the fibers in physiological environments. This is essential for assessing their suitability for biomedical applications such as tissue engineering and drug delivery. Interactions with external stimuli can be analyzed to examine how fibers respond to external stimuli such as mechanical forces, temperature changes, and biochemical signals. This includes studying phenomena like strain-stiffening, swelling, and conformational changes. Modification and functionalization include aspects to investigate methods for modifying and functionalizing fibers to tailor their properties for specific applications. This may involve surface modification, chemical conjugation, or incorporation of bioactive molecules. Integration into biomaterials can be studied to explore strategies for incorporating fibers into biomaterials and scaffolds to enhance mechanical strength, biological functionality, and tissue regeneration. This includes studying fabrication techniques and scaffold architecture. Biomedical applications can be evaluated by assessing the potential biomedical applications of biological fibrous materials, including tissue engineering, wound healing, drug delivery, and medical implants. By analyzing these aspects, researchers can gain a comprehensive understanding of biological fibrous materials and harness their unique properties for various biomedical and biotechnological applications.

In biomedical applications, one important aspect is the diagnosis of the disease state and progression. The progression of many common and fatal diseases, including various cancers, neurodegenerative disease and heart disease are characterized by changes in tissue microstructure. Fibrosis, a common byproduct of such diseases, is characterized by buildup of extracellular matrix (ECM). The ECM is largely made of collagen, the most abundant ordered structural protein in the human body. These fibers exhibit birefringence, as their form and molecular composition permit the selective interaction with polarized light. Further tissue and cellular components, such as cell nuclei and other biological fibers (e.g. elastin, amyloids, actomyosin) have demonstrated this anisotropy, so there is large potential to examine their structure within disease progression with this optical property.

The disclosure provides a method and platform for imaging and analyzing the microstructure of fibrous materials. The platform employs a polarized light microscopy imaging system enhanced by a colorimetric photonic crystal. Examples of photonic crystals in nature include wings, shells, leaves, feathers, and skins, i.e., microscopically structured surfaces that are capable of interfering with visible light, often referred to as “structural coloration”. Specific cases of such organisms include butterflies of the Morpho, Junoia, Parides, and Papilio genus, Jewel beetles and other beetles and weevils, several organisms of the Cephalopoda class (e.g., squid, cuttlefish, octopus), several plants including those of the genus Selaginella, diatoms, the feathers of peacocks, birds of paradise, hummingbirds, and the Eurasian Jay, among many others. In the feathers of birds and the scales of butterflies, interference can be created by a range of photonic mechanisms, including diffraction gratings, selective mirrors, photonic crystals, crystal fibers, matrices of nanochannels, and proteins that can vary their configuration.

A prominent example of structural coloration is the Blue Morpho butterfly wing. Its dorsal surface is covered with a double layer of scales, comprised of parallel longitudinal striations with periodic separation between ridges. Vertically stacked chitin lamellae that make up the ridges produce a multilayer interference effect that increases the reflectivity of the wing and the purity of the reflected color. The diffraction-grating arrangement of its longitudinal ridges separates light into different wavelengths depending on incident angle. This means redder hues (620-750 nm) are seen at larger angles than bluer wavelengths (380-500 nm). Another profound optical property of the Morpho wing is its strong ability to polarize light. The directionality of the longitudinal ridges derives a sensitivity to the transverse electric (TE) and transverse magnetic (TM) modes of light propagation where the electric and magnetic fields are parallel or perpendicular to the gratings respectively. The reflection spectra of the Morpho menelaus are shown to shift from the blue regime when illuminated by a transverse electric (TE) wave towards green when illuminated with a transverse magnetic wave. Further, when imaged between crossed polarizers the scales appear darker when the orientations of the ridges coincide with the polarizer or analyzer, but the scales are bright when oriented at ±45°. This means that the scales introduce a phase delay unto the light that produces a high degree of elliptical polarization. Thus, the structural anisotropy of the Morpho wing gives rise to the ability to map the polarization state of light to unique optical responses. In the examples described herein, we employ these powerful optical properties of the Morpho butterfly wing: (1) strong reflection of visible light at selected frequencies and (2) polarization-sensitive colorimetry to arrive at Morpho-enhanced polarized light microscopy. This offers a sensitive, simplified, and noninvasive photonic crystal-centric approach for imaging structural anisotropy in fibrous materials that can be implemented in diagnostic imaging. Existing techniques face tradeoffs in time, cost, and precision. The inventive imaging platform addresses the tradeoffs faced by existing tissue-imaging modalities, offering a rapid, contact-free, and stain-free approach that significantly reduces experimental complexity by miniaturizing sophisticated optics onto a single, micro- and nanostructured surface. As an illustrative example, the polarization-sensitive structural color of the Morpho butterfly wing is used to probe collagen arrangement in breast cancer tissues. Based on this example, it will be apparent to those of skill in the art that this platform can be extended to many other fibrotic diseases and photonic crystal geometries.

The exemplary implementation of the inventive imaging platform employs a conventional glass microscope slide supporting a section excised from a Morpho butterfly wing. This section is adhered onto a glass slide. A thin cover slip is adhered on top of the wing section to secure the wing section and separate the scales from the external environment. Then, a slide containing the specimen of interest is placed on top of this Morpho butterfly wing slide. This assembly is imaged with a polarized light microscope. The fibrous material and photonic crystal can be imaged in reflection mode (episcopic), or in transmission mode (diascopic) of the polarized light microscope.

The platform implements the physical process of structural color, in which light interacts with a micro-/nanostructured surface that strongly reflects back wavelength-specific colors. The scales of the Morpho butterfly wing are composed of nanostructures that reflect blue light when it is illuminated by white light—such as the light emitted from a light microscope. These nanoscale features are also anisotropic, thus they extend differently across different spatial directions. This results in a color sensitivity to the incident angle or polarization of light. When interfaced with fibrous biological tissue—which changes the polarization state of light based on its microstructural properties—the butterfly wing will map tissue microstructure onto a reflected color and intensity. The result is a histological image in which distinct biological properties corresponding to fiber alignment, density and orientation are distinguished by specific color and intensity responses. The platform enables enhanced visualization of the microstructure and properties of fibrous materials such as biological tissues and how they may change through different mechanical and/or biological processes. This is accomplished by mapping fiber alignment and density in the fibrous materials onto specific colors and intensities in a diagnostic manner.

The disclosure further provides a method for quantitatively evaluating the microstructure of fibrous materials. This method involves mounting the fibrous material onto a slide, placing the slide onto another slide equipped with a colorimetric photonic crystal on a microscope stage, capturing images of the fibrous material using polarized light transmitted through and/or reflected from the slides, and analyzing the intensity profile of the images using a mathematical model. The fibrous material and photonic crystal can be imaged in reflection mode (episcopic), or in transmission mode (diascopic) of the polarized light microscope.

An exemplary method for analyzing tissue color response is detailed in the disclosure herein. It involves harnessing structural color generation, achieved through the application of polarized light microscopy combined with the intricate structure of a Morpho butterfly wing. The scales that cover the surface of the Morpho butterfly wing consist of micro- and nanoscale geometry that behave as a diffraction grating, generating blue light at normal incidences. The Morpho wing also displays an anisotropic color response depending on how light is polarized when it interacts with the geometric patterns on the wing surface. As collagen undergoes distinct alignment changes across various disease stages, it induces optical anisotropy upon light interaction, resulting in polarization of the light by these fibers. Due to the polarization-sensitive optical properties of the Morpho wing and collagen fibers, the fibrotic tissue interfaced with the Morpho wing can be imaged. This method produces a color and intensity response specific to the alignment, density and orientation of the collagen in the tissues.

### Imaging Platform

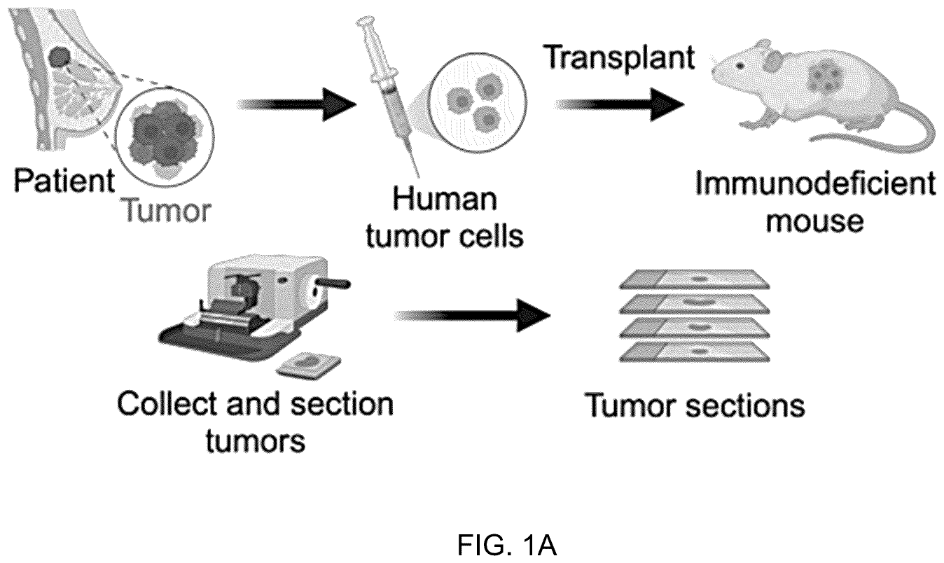

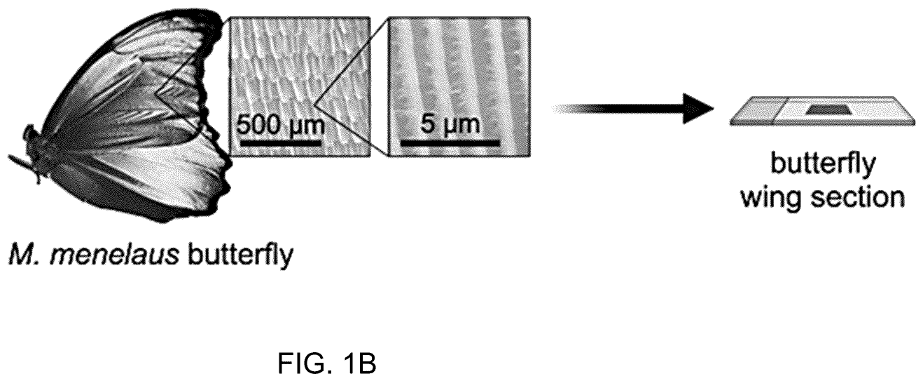

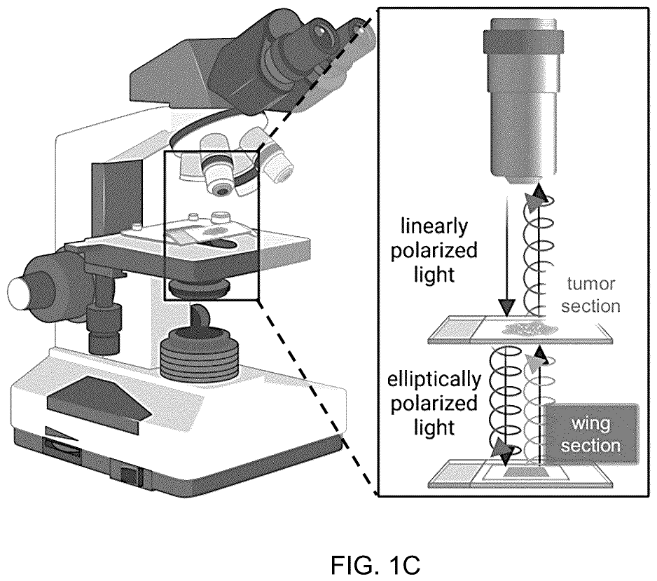

FIGS. 1A-1C schematically illustrate the principle of Morpho-enhanced polarized light microscopy according to the inventive scheme. As one exemplary system of fibrous biological tissue, we studied patient derived xenograft (PDX) models for treatment-naïve triple-negative breast cancer (TNBC) subtypes (FIG. 1A). For this study, we select two tissue types based on stiffness, which exhibit dense or sparse collagen content, respectively. The paraffin-embedded samples were sectioned at 3 μm thickness. FIG. 1B shows a photograph and scanning electron micrographs of the studied Morpho butterfly wing, which is placed onto a glass microscope coverslip and covered with an additional glass coverslip to protect the scales upon imaging. Morpho-enhanced polarized light microscopy is shown in FIG. 1C, where the Morpho butterfly wing and tissue sections shown in parts a and b are placed on top of each other on the microscope stage. Importantly, this means that no direct contact between the Morpho wing and the tissue of interest is needed for this technique, as it relies on polarized light-matter interactions in the far field. The inset of FIG. 1C shows schematically how linearly polarized light is incident upon the tissue of interest. Upon traversing the fibrous tissue sample, the polarized light will gain a degree of ellipticity. The strong optical anisotropy of the Morpho butterfly wing will then further alter the ellipticity of the light in reflection before it passes through the tissue section a second time and enters an analyzer orthogonal to the orientation of incident linearly polarized light. Thus, this method enables the specific enhancement of the optical anisotropy of the fibrous tissue by leveraging the optical anisotropy of the Morpho butterfly wing. Subsequently, changes in the density, organization, and orientation of collagen fibers in the tissue of interest can be quantitatively assessed with Morpho-Enhanced polarized light microscopy, while they would fall below detection thresholds if the Morpho wing were absent in conventional polarized light microscopy. Because this method is contact free and label free, assessment of the microstructural properties of the fibrous biological tissue occurs without necessitating any staining procedures and the Morpho butterfly wing can be reused over many analysis cycles for a multitude of different tissue sections, significantly accelerating and democratizing experimental procedures to assess biological tissue microstructure.

### Theoretical Model

Within an identified region of interest in an imaged tissue section, the average pixel intensity was acquired for each angle of rotation. The resulting intensity profile of the image series was fit using a mathematical model based on Jones calculus, which assumes fully polarized light excitation and linear optical components, to characterize light propagation in these optically anistropic regions of the sample of interest. In the example case studied here, regions containing collagen in the tissue of interest exhibit optical anisotropy.

All optical components in Jones calculus are described by Jones matrices. The Jones matrices of the horizontal (oriented along the x-axis) and vertical (oriented along the y-axis) polarizers respectively are:

$${P}_{x} = ( \begin{matrix}1 & 0 \\ 0 & 0\end{matrix} ) and {P}_{y} = ( \begin{matrix}0 & 1 \\ 0 & 0\end{matrix} ) \qquad ( 1 )$$

The Jones matrix of a wave retarder rotated by an angle ψ in the counterclockwise direction and induces a phase shift δ along its optical axis between the two orthogonal polarizers is given by:

$$J ( δ , ψ ) = ( \begin{matrix}cos ψ & - sin ψ \\ sin ψ & cos ψ\end{matrix} ) \times ( \begin{matrix}{e}^{i δ / 2} & 0 \\ 0 & {e}^{- i δ / 2}\end{matrix} ) \times ( \begin{matrix}cos ψ & sin ψ \\ - sin ψ & cos ψ\end{matrix} ) \qquad ( 2 )$$

As the Morpho wing and tumor sections (M+T) are simultaneously rotated together in the counterclockwise direction by the rotation angle θ between the two fixed orthogonal polarizers, the rotation of the M+T can be described as ψ=φ+θ, where φ is the optical axis orientation of the Morpho wing and tissue section together. Thus, the Jones matrix can be modified to:

$${J}_{M + T} ( δ , ϕ + θ ) = ( \begin{matrix}cos ( ϕ + θ ) & - sin ( ϕ + θ ) \\ sin ( ϕ + θ ) & cos ( ϕ + θ )\end{matrix} ) \times ( \begin{matrix}{e}^{i δ / 2} & 0 \\ 0 & {e}^{- i δ / 2}\end{matrix} ) \times ( \begin{matrix}cos ( ϕ + θ ) & sin ( ϕ + θ ) \\ - sin ( ϕ + θ ) & cos ( ϕ + θ )\end{matrix} ) \qquad ( 3 )$$

The microscope lamp emits unpolarized light, which passes through an input linear polarizer before entering the sample. The light incident on the Morpho+Tissue arrangement is thus described as the Jones vector by Eₓ=Pₓ·E₀, where E₀ is the electric field vector of light emitted by the lamp and Eₓ is the horizontally polarized light that exits the input linear polarizer. When plane-polarized light (Eₓ) enters the Morpho+Tissue arrangement, the electric field reflected off the tissue surface is described by E_{M+T}=J_{M+T}(δ, φ+θ)·Eₓ. This light then passes through the vertically oriented analyzer (P_{y}) such that the transmitted light captured by the microscope camera is Eₒᵤₜ=P_{y}·E_{M+T}. Using I~|Eₒᵤₜ|², the transmitted light intensity is calculated to produce the second-power sinusoidal intensity profile:

$$I ( θ ) = {I}_{0} {sin ( 2 ϕ + 2 θ )}^{2} sin {( \frac{δ}{2} )}^{2} \qquad ( 4 )$$

Where I₀=1, equivalent to the normalized grayscale pixel value of the incident lamp intensity. The phase delay stemming from linear birefringence δ is given by:

$$δ \approx \frac{2 π}{λ} Δ nd \qquad ( 5 )$$

Where λ is the wavelength of incident light, Δn is the local birefringence, and d is the tissue thickness. Previous studies have established that increased presence of collagen fibers corresponds to an increase in collagen birefringence (Δn). As the incident wavelength and sample thickness (3 μm) are fixed parameters, the intensity profile equation provides a direct measure of the relative fiber orientation and fiber density within a region.

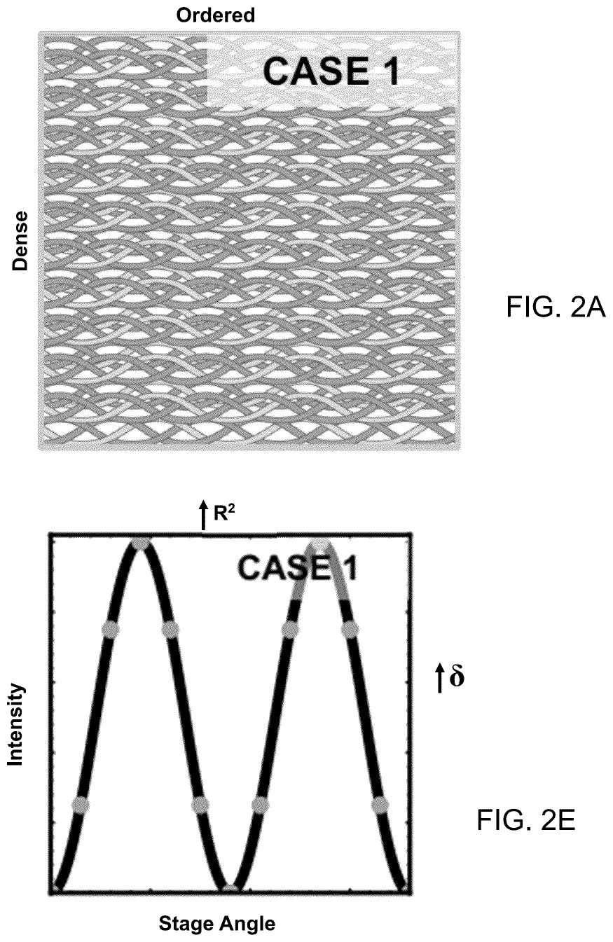

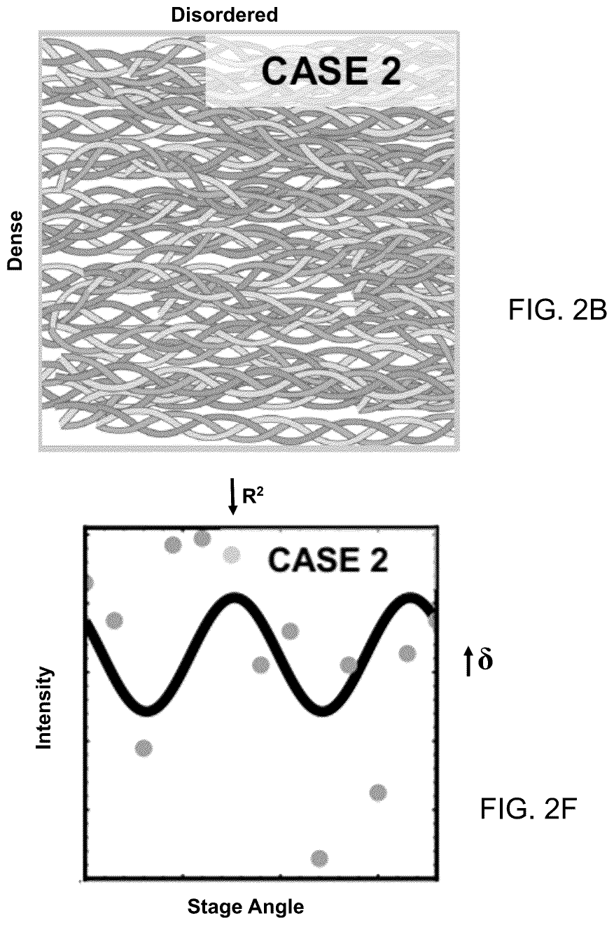

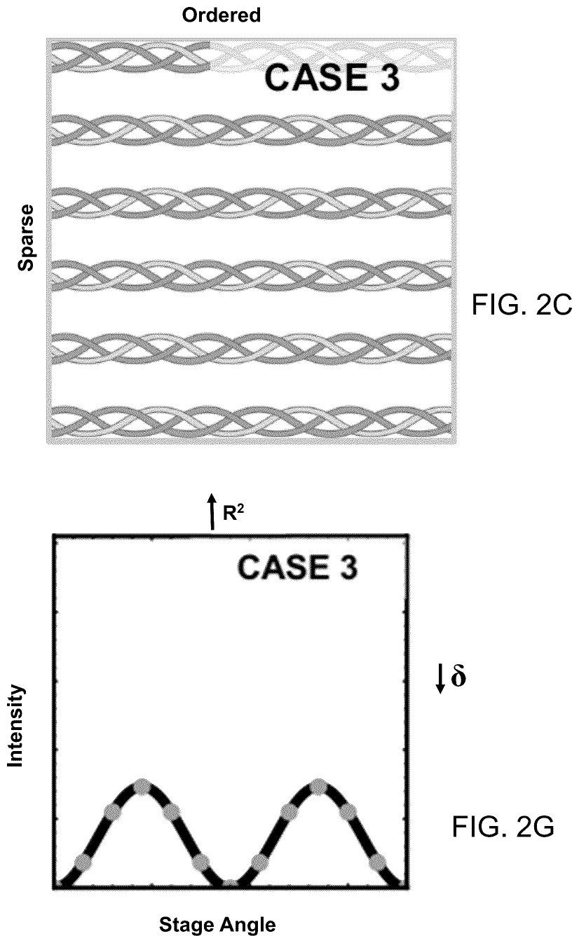

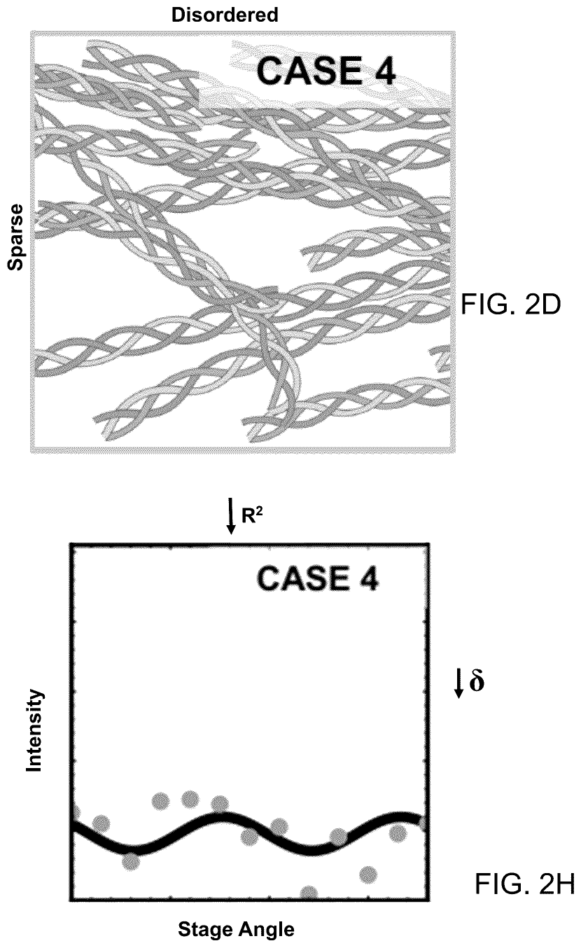

FIGS. 2A-2H describe our Jones calculus model (Equation 4) which quantifies the fibrous properties of biological tissue when interfaced with the Morpho butterfly wing. We divide our observations into 4 distinct cases—note that realistic cases may lie between these extreme scenarios of dense vs. sparse collagen content, directly related to δ in Equation 4 and ordered vs. disordered collagen arrangement, directly related to the R² fit value of Equation 4.

An important behavior to note is that as the Jones fit for the intensity profile decreases in certainty with lower R² values, the breadth of estimated δ values also increases. This is seen in the simulated Case 2 (FIGS. 2B, 2F) where we theorize an extreme case of density and disorder. The MATLAB® Jones fit approximates δ=48.048°±76.524°, despite the inserted pixel intensities having been derived for a δ=90° value. When comparing the δ values for Case 1 and Case 2, we cannot definitely ascertain one being more dense than the other given the wide 95% confidence interval for Case 2.

### EXPERIMENT DETAILS

### Characterization of Optical Anisotropy in the Morpho Butterfly Wing

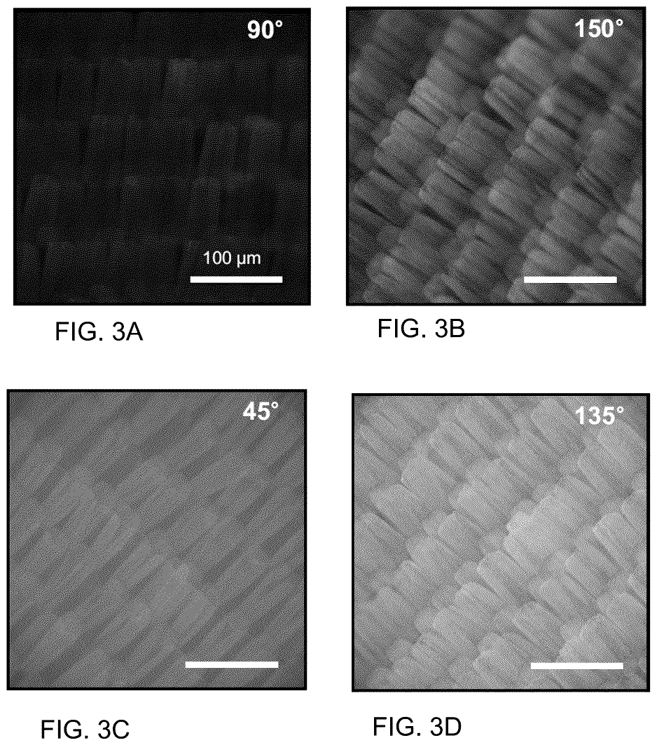

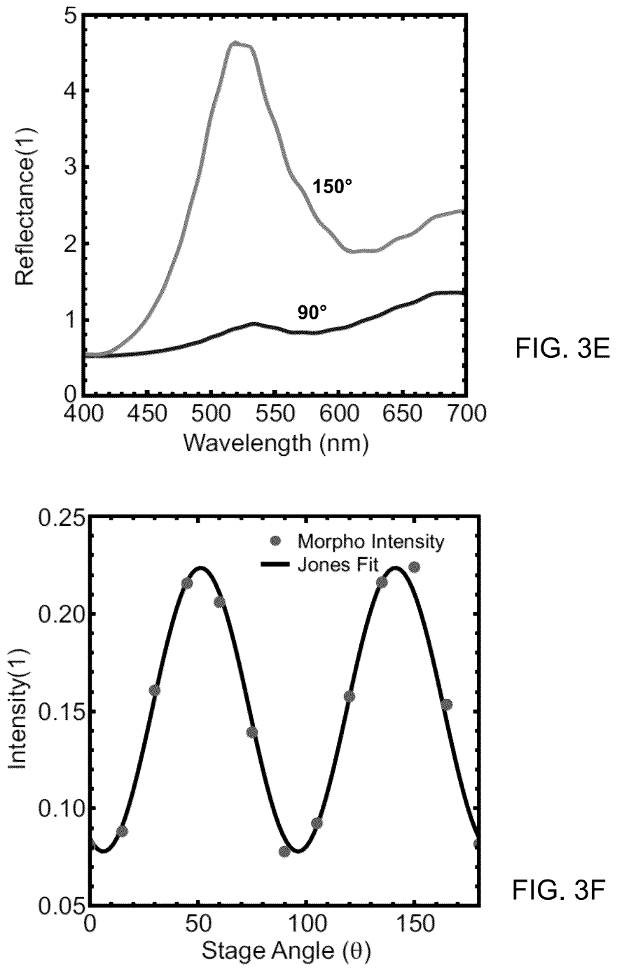

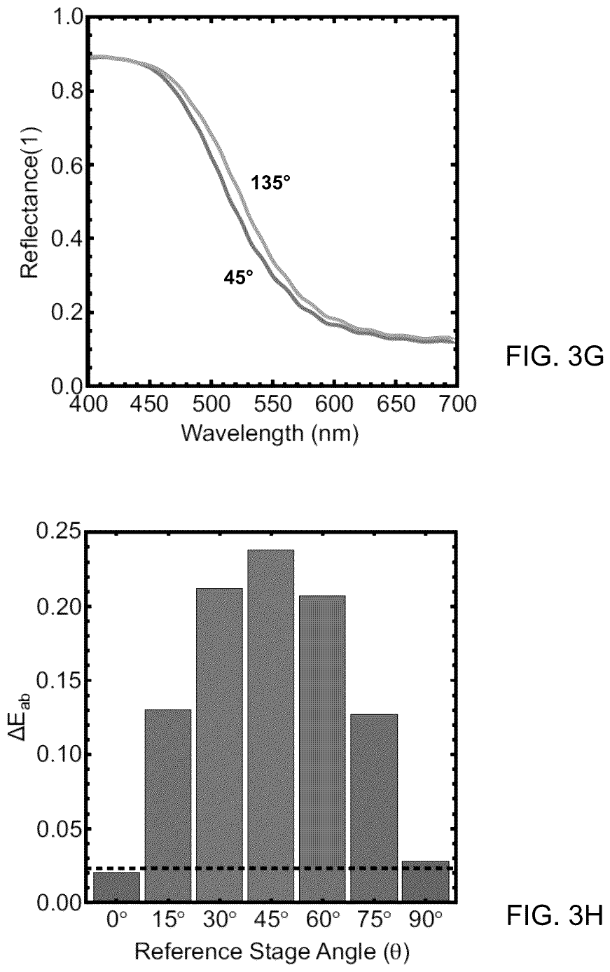

Example images for linear (circular) excitation are shown in FIGS. 3A-3D, respectively, where the corresponding reflectance spectra were recorded in FIGS. 3E and 3G. The Jones calculus model (Equation 4) presented in this work was applied to the Morpho butterfly wing in FIG. 3F.

FIGS. 3A-3F characterize the anisotropic optical properties of the Morpho butterfly wing upon linearly and circularly polarized light excitation with a linear analyzer. The Morpho wing section was rotated counterclockwise from 0°-180° in 15° increments. Two series of imaging took place, the first in which the sample was illuminated with horizontally polarized light and an orthogonal analyzer, and for the latter a quarter-wave retarder was introduced to the light path for circularly polarized light excitation and an identical linear analyzer. These images were acquired with NIKON® NIS-Elements Basic Research Software.

Illumination and analysis with orthogonal linear polarizers resulted in the highest reflectance of the Morpho butterfly wing at 150° and lowest at 90° stage orientation (images in FIGS. 3A-3D). For each image of the wing section, the pixel intensity was averaged and normalized by maximum pixel intensity (255) in MATLAB®. The average intensity for each orientation was plotted and fitted to the Jones Calculus model with the MATLAB® built-in nonlinear least squares function in the Curve-Fitting tool (FIG. 3F). The Jones fit yielded a retardance (δ) of 47.08°±3.707°. Given the high accuracy of the fit (R²=0.9890) the following results were presumed to accurately model the linear behavior of the Morpho wing as a wave retarder.

Spectra of the Morpho wing at the recorded orientations producing its minimum (90°) and maximum (150°) intensities under linearly polarized light illumination were acquired with an IsoPlane 320A Spectrometer (FIG. 3F). At 150° the normalized reflectance peaks at 4.637 at 518.4 nm. At 90° this reflectance is damped to 0.8784, with the peak intensity in the green regime being 0.9450 at 534.0 nm. The highest reflectance at this orientation was observed at 691.6 nm to be 1.355.

Studies conducted on the Morpho wing scales have shown that their longitudinal ridges form a diffraction structure that polarizes light. The scales do not maintain the same orientation throughout the wingspan and the optical behavior of the wing section is sensitive to the position in which it is adhered to the glass slide. Therefore, this fit encompasses the global response of the Morpho wing in this experiment. The Jones calculus fit revealed the derived optical axis (φ) of this section of the Morpho wing to be φ_{Morpho}=5.798°±2.237° and the extrapolated intensity maxima and minima from this fit can be found at orientations 50.71° and 5.455°, respectively. A birefringent material achieves its highest reflection intensity when its optical axis is oriented ±45° between orthogonal polarizers and its lowest reflection intensity when its optical axis is aligned with either polarizer. Thus, these results are in line with what is to be expected in conventional polarized light microscopy.

The Commission Internationale de l'Eclairage (CIE) system of colorimetry determines the amount of the three tristimulus values corresponding to the three primary colors red, green, and blue that are needed to be mixed to produce a specific color through the execution of color-matching functions. To quantify the colors generated in response to a stimulus, one can convert the obtained chromaticity coordinates from the 1931 CIE XYZ color space, which specifies color according to the visual system of the eye, to the 1976 CIE Lab color space, where L* indicates perceptual lightness, and a* and b* represent the color ranges of human vision: red to green and blue to yellow. By taking the non-linear transform of the XYZ color coordinates, one can enter the CIE Lab system of color specification.

Images acquired of the Morpho wing upon circularly polarized illumination with a linear analyzer displayed anisotropic color responses. The difference in color detectable by the human eye was quantified with the 1976 CIE Lab color discrimination factor, for which we calculate the Euclidean distance between two colors in the 1976 CIE Lab colorspace with chromaticity coordinates L₁,a₁,b₁ and L₂,a₂,b₂:

$$Δ {E}_{ab} = \sqrt{{( {L}_{2} - {L}_{1} )}^{2} + {( {a}_{2} - {a}_{1} )}^{2} + {( {b}_{2} - {b}_{1} )}^{2}} , \qquad ( 7 )$$

ranging from 0 to 1 with the Just Noticeable Difference discernable by the human eye (JND) at ΔE_{ab}=0.023. Originally acquired in the RGB colorspace, for each image the mean RGB value of nonblack pixels (pixels for which the Red, Green, and Blue channels were valued greater than zero) was converted to yield chromaticity coordinates in the 1976 CIE Lab colorspace. The color discrimination function was then implemented to calculate the color difference between images taken at mutually orthogonal orientations of the Morpho section (FIG. 3H). Nonzero color difference values were observed for orientations 0-90°, demonstrating that the Morpho wing breaks C₄ rotational symmetry.

We observe a peak color discrimination (ΔE_{ab}=0.2382) between the Morpho wing at 45° and 135°. As the color difference tapers off in a somewhat normal distribution, this birefringence effect is minimized at 0° and 180°, resulting in the lowest color differences comparing the color seen at 0° and 90° to the color at the orthogonal position (ΔE_{ab}=0.0203487, 0.02783). This coincides with the analyzer being oriented at 90°, selecting out components of light that coincide with the vertical axis. Thus, the effects of form birefringence should be minimized when the optical axis of the Morpho wing (φ_{Morpho}=5.798°±2.237°) is closely in line with the polarizer and/or analyzer.

Additionally, when the Morpho wing is oriented at 135° the grating lines are oriented 45° relative to the direction of rotation of the incident left-handed circularly polarized light's electric field vector. In turn, at a respective 135° orientation, this handedness is diagonally opposite to the direction of circular polarization, minimizing the interaction between the scales and the incident polarized light. This is supported by the acquired spectra of the Morpho wing oriented at 45° and 135° under circularly polarized light illumination where we see the higher reflectance at 135° (FIG. 3G). The reduced micro- and nanostructure interference means the color presented by the Morpho wing is predominantly derived from the melanin pigments within the scales rather than the nanostructures, which is why we see a significant color difference for these two orientations.

### Morpho-Enhanced Polarized Light Microscopy for Characterization of Murine Breast Cancer Tissue

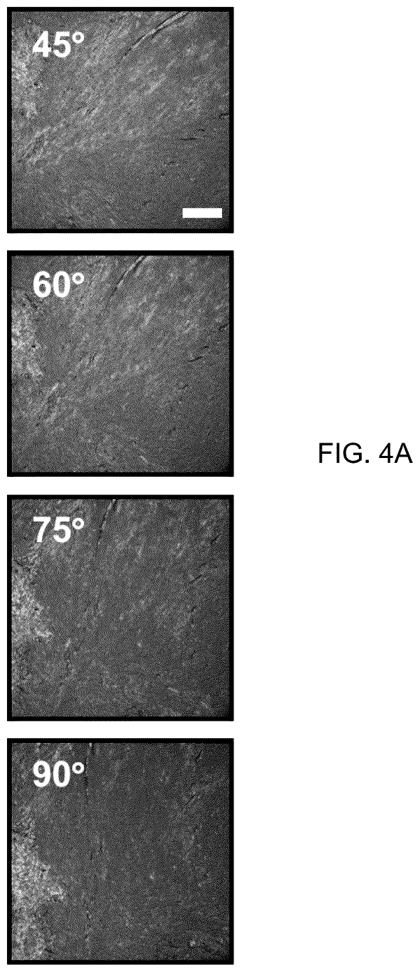

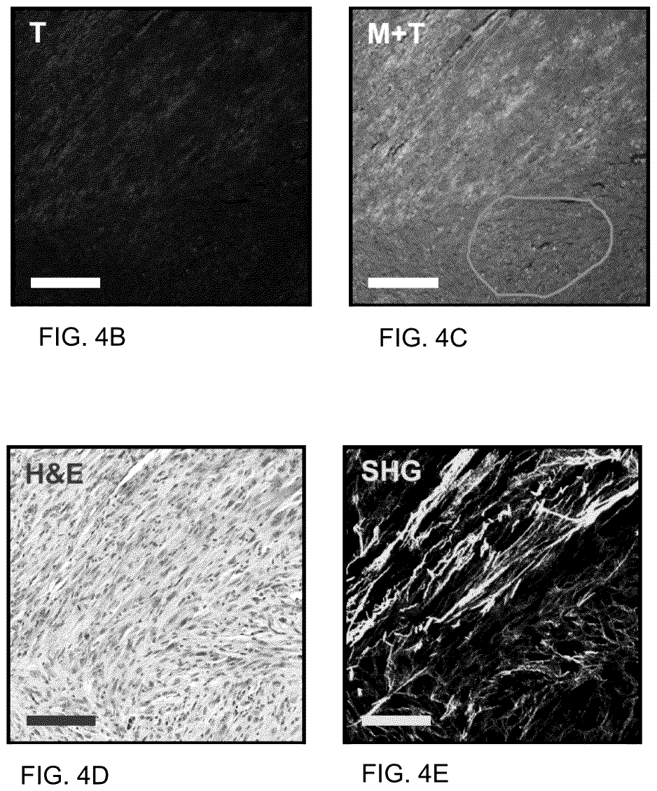

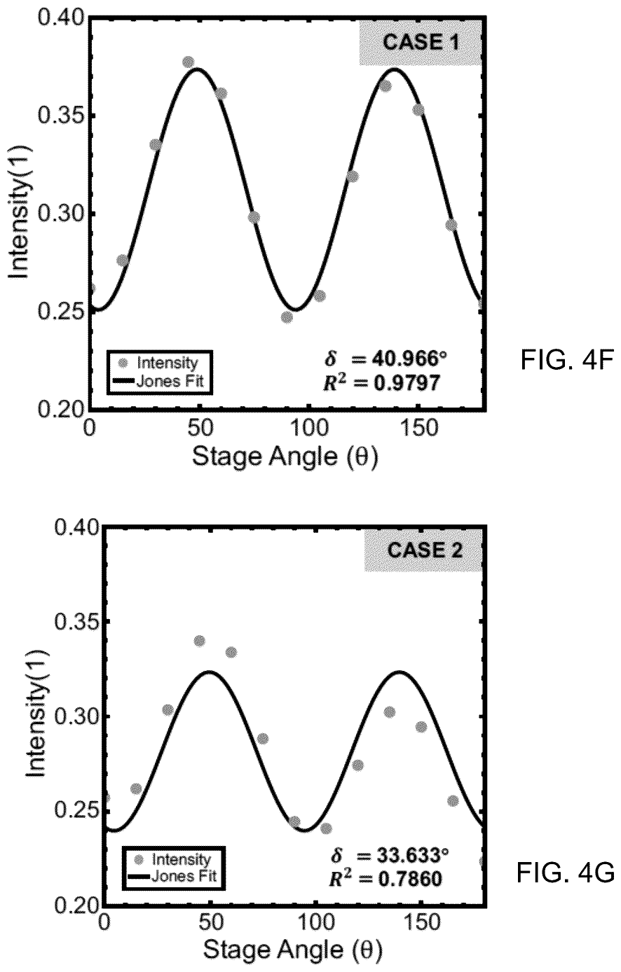

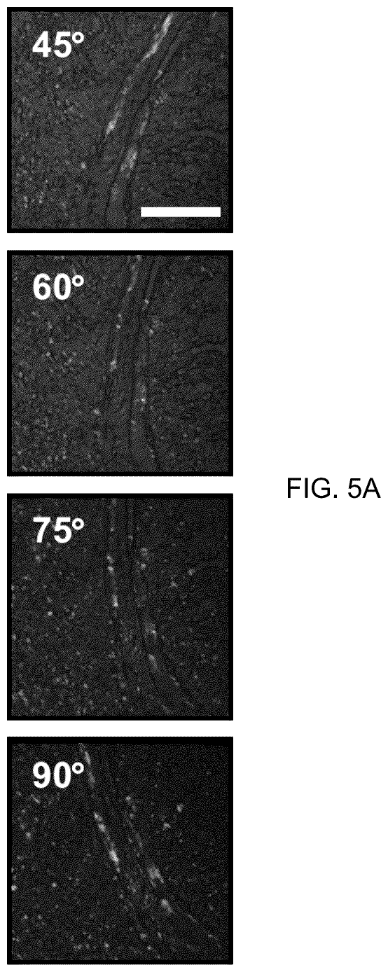

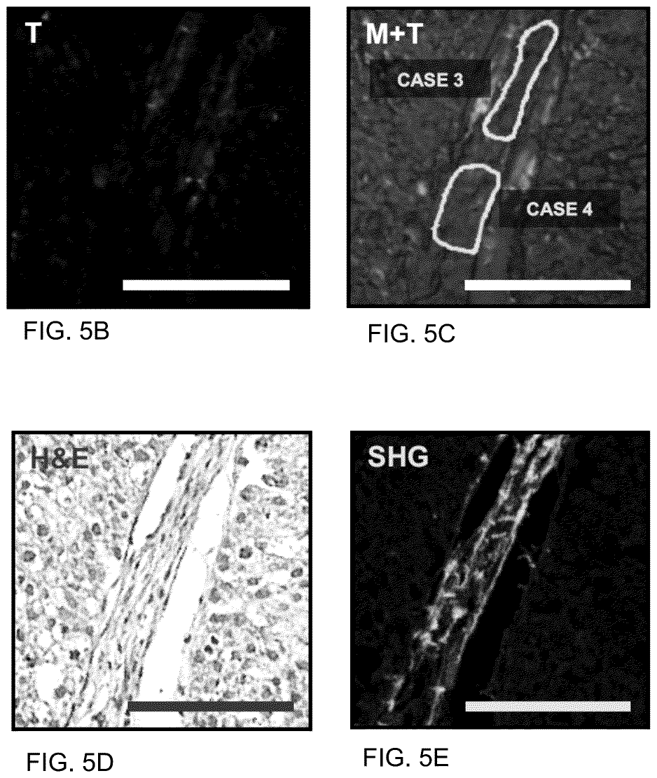

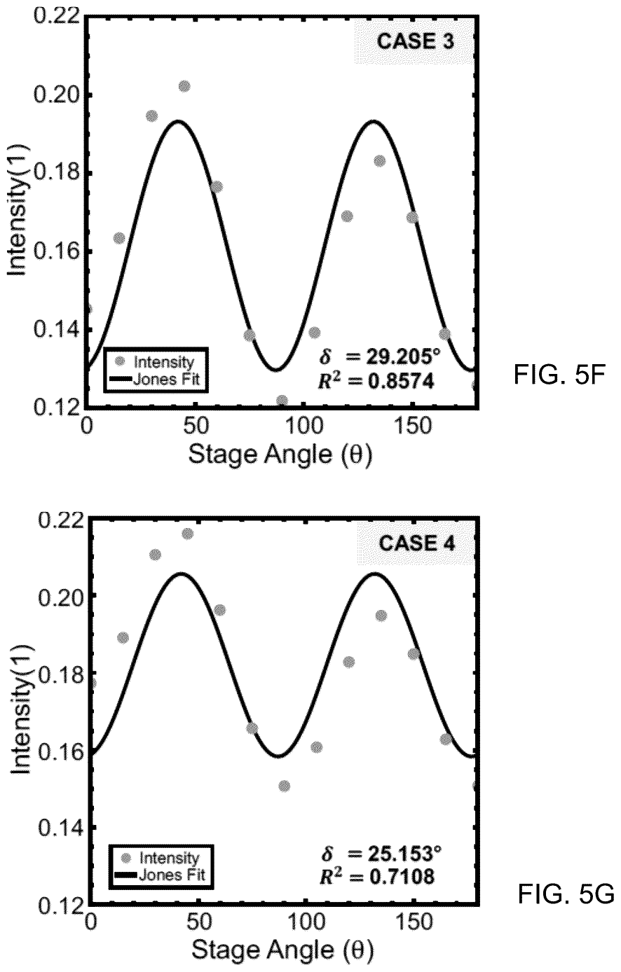

Collagen dense and collagen sparse tumor tissue sections were imaged with and without the Morpho wing between crossed linear polarizers undergoing the same 15° increments of rotation for observation of intensity variations due to optical anisotropy. The same sections were then deparaffinized, stained with H&E and imaged in transmission with the same polarized light microscope. H&E images were adjusted with NIS Element's built-in white balance function for color balancing. The H&E-stained sections were then imaged with SHG microscopy to probe for collagen content. The resulting images of the collagen dense and collagen sparse tumor tissue sections are shown in FIGS. 4A-4G and 5A-5G, respectively. From the optical behavior and pathologist-interpreted H&E and SHG images, Regions of Interest (ROIs) corresponding to Cases 1-4 (FIGS. 2A-2D) were identified in the collagen dense and collagen sparse tumor sections. Jones Calculus analysis (Equation 4) was implemented for these ROIs in images acquired when the unstained tumor tissue section was interfaced with the Morpho wing (M+T).

FIGS. 4A-4G provide a quantitative analysis of the microstructural properties in the studied collagen dense tissue section for two separate ROIs representing examples of Case 1 (dense, ordered) and Case 2 (dense, disordered) described in FIGS. 2A-2B. Jones calculus analysis (Equation 4) resulted in corresponding δ values for the ROI representing Case 1 (40.966°±4.343°) and Case 2 (33.633°+12.743°), respectively. As predicted by our model (FIGS. 2E-2F), Jones calculus analysis results in a higher R² value for Case 1 (0.9797) than Case 2 (0.7860), indicating a comparatively higher collagen alignment for Case 1.

FIGS. 5A-5G quantitatively analyze the microstructural properties of the studied collagen sparse tissue section for two separate ROIs representing examples of Case 3 (sparse, ordered (FIG. 2C)) and Case 4 (sparse, disordered (FIG. 2D)). Two regions of interest along the fibrillar strip were imaged (FIG. 5B). Implementing Jones calculus analysis (Equation 4), the MATLAB optimization algorithm arrived at a comparably higher δ value for Case 3 than Case 4 (29.205°±8.583°, 25.153°±11.4935°) (FIGS. 5F, 5G). The R² value for this data also decreased from Case 3 to Case 4 (0.8574, 0.7108) (FIGS. 5F, 5G). Given that R² represents how closely the true intensity values align with those approximated with the Jones calculus model and the Jones Calculus model assumes completely linear optical elements, high R² values correspond to higher degrees of anisotropic alignment within the region of interest. The increased amount of collagen fibers presents an increased amount of retardance δ. Thus, higher δ indicates higher fiber density with respect to area in the studied Case 3 and 4 ROIs (FIGS. 5A-5G) compared to Cases 2 and 3 (FIGS. 4A-4G) (Equation 5).

It is worth noting that collagen is not the only fibrous tissue or component that exhibits form birefringence. Therefore, this Jones calculus model (Equation 4) is not specific to collagen. The disclosure herein provides one example using the intentionally selected regions identified to be predominantly collagen from pathologist assessment of the SHG micrographs and H&E stains. As will be readily apparent to those of skill in the art based on the foregoing examples, the platform and the method can be applied to investigate the optical behavior of other extracellular fibers and various material microstructure. The inventive scheme provides a low cost, label-free imaging approach that enables evaluation of microstructure of materials of both natural and synthetic forms. The application in biological materials enables the imaging and quantitative assessment on changes in tissue microstructure that occur in various diseases.

1. A method for quantitative assessment of the microstructure of a fibrous material comprising:
mounting the fibrous material on a first slide;
disposing the first slide on top of a second slide comprising a colorimetric photonic crystal mounted thereon on a microscope stage;
imaging the fibrous material using polarized light projected through the slides; and
analyzing the intensity profile of the image by a mathematical model.

2. The method of claim 1, wherein the colorimetric photonic crystal is derived from the structural color of an organism.

3. The method of claim 1, wherein the colorimetric photonic crystal is derived from a Morpho butterfly wing.

4. The method of claim 1, wherein the fibrous material is in natural form or in synthetic form.

5. The method of claim 4, wherein the fibrous material is a biological tissue.

6. The method of claim 5, wherein the biological tissue comprises fibrous tissue.

7. The method of claim 1, wherein the imaging is performed in reflection mode of the polarized light microscope.

8. The method of claim 1, wherein the imaging is performed in transmission mode of the polarized light microscope.

9. The method of claim 1, wherein the mathematical model is based on Jones calculus.

10. A platform for imaging a fibrous material, comprising:
a light microscope having a microscope stage, the microscope having a light source configured to project polarized light through one or more slides disposed on the stage;
a first slide comprising a colorimetric photonic crystal;
a second slide for retaining a sample of the fibrous material; and
the second slide is disposed on top of the first slide so that when polarized light is projected through the slides, and the first slide operates as an anisotropic material whereby fiber alignment within the fibrous material is imaged and differentially illuminated.

11. The platform of claim 10, wherein the colorimetric photonic crystal is derived from the structural color of an organism.

12. The platform of claim 11, wherein the colorimetric photonic crystal is derived from a Morpho butterfly wing.

13. The platform of claim 10, wherein the fibrous material and the colorimetric photonic crystal are imaged in reflection mode (episcopic) of the polarized light microscope.

14. The platform of claim 10, wherein the fibrous material and the colorimetric photonic crystal are imaged in transmission mode (diascopic) of the polarized light microscope.
